(19) United States
(12) Patent Application Publication
(10) Pub. No.: US 2023/0279036 A1
(43) Pub. Date: Sep. 7, 2023

(54) Salts of Remdesivir

(71) Applicant: Apotex Inc., Toronto (CA)

(72) Inventors: Fabio E.S. Souza, Brantford (CA); Fatemeh Mohammadpourmir, Brantford (CA); Alexander J. Stirk, Brantford (CA); Avedis Karadeolian, Brantford (CA); Allan W. Rey, Brantford (CA)

(21) Appl. No.: 18/018,308

(22) PCT Filed: Jul. 20, 2021
(86) PCT No.: PCT/CA2021/051003

§ 371 Date: Jan. 27, 2023

Related U.S. Application Data
(60) Provisional application No. 63/093,481, filed on Oct. 19, 2020.
(60) Provisional application No. 63/057,341, filed on Jul. 28, 2020.

(51) Int. Cl. C07F 9/6561

(52) U.S. Cl. CPC C07F 9/6561; C07B 2200/13

(57) ABSTRACT

The present invention provides novel salts of remdesivir and crystalline forms thereof. Specific salts of remdesivir provided by the present invention include napsylate, tosylate, hydrochloride, phosphate, maleate, and oxalate. Also provided are pharmaceutical compositions including the remdesivir salts and crystalline forms thereof, the use of these salts in the treatment of a viral infection, and methods of treating viral infections using the same, and in particular, a viral infection caused by Severe Acute Respiratory Syndrome coronavirus 2 (SARS-CoV-2).

### CROSS REFERENCE TO RELATED APPLICATIONS

This application is the United States national phase of International Patent Application No. PCT/CA2021/051003 filed Jul. 20, 2021, and claims priority to U.S. Provisional Patent Application No. 63/057,341 filed Jul. 28, 2020, and U.S. Provisional Patent Application No. 63/093,481 filed Oct. 19, 2020, the disclosures of which are hereby incorporated by reference in their entireties.

### BACKGROUND OF THE INVENTION

### Field of the Invention

The present invention is directed to novel salts of remdesivir, crystalline forms thereof, pharmaceutical compositions containing these salts, their use in the treatment of viral infections, and methods of treating viral infections.

### Description of Related Art

Remdesivir (1), or (S)-2-ethylbutyl-2-(((S)-(((2R,3S,4R,5R)-5-(4-aminopyrrolo[2,1-f][1,2,4]triazin-7-yl)-5-cyano-3,4-dihydroxytetrahydrofuran-2-yl) methoxy)(phenoxy)phosphoryl)amino)propanoate, is a nucleotide analog that is reported to exhibit antiviral properties against Arenavirdae, Coronaviridae, Filovirddae, and Paramyxoviridae viruses. Remdesivir is the active ingredient in VEKLURY® indicated for adults and some pediatric patients for the treatment of coronavirus disease 2019 (COVID-19) requiring hospitalization.

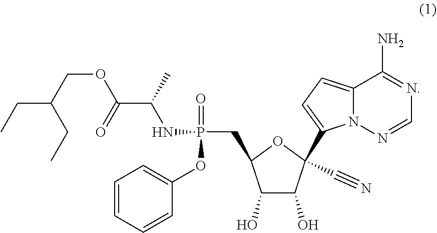 [SMILES: CCC(CC)COC(=O)[C@H](C)N[P@](=O)(C[C@H]1O[C@@](C#N)(c2ccc3c(N)ncnn23)[C@H](O)[C@@H]1O)Oc1ccccc1]

In addition to crystalline Forms I to IV and mixtures thereof, a maleate salt of remdesivir, and crystalline Form I thereof, is reported in WO 2018/204198 A1.

According to the European CHMP Summary on Compassionate Use for Remdesivir Gilead (EMEA/H/K/5622/CU), the neutral drug substance remdesivir is administered to patients intravenously. However, due to the limited aqueous solubility of remdesivir, the beta-cyclodextrin derivative Betadex sulfobutyl ether sodium (SBECD) must be used as a solubilizing agent in the formulation. Due to poor oral bioavailability of the known crystalline form(s) of remdesivir, the drug is administered intravenously, which raises concerns about limits to widespread distribution of the medication to the broader public, if it is shown to be safe and effective in the treatment of COVID-19. Additionally, WO 2019/014247 A1 discloses that remdesivir is chemically unstable in an aqueous environment.

The solubility of individual salt and crystalline forms of a drug substance in an aqueous environment often correlates with their relative bioavailability, since the manner in which the salt or crystalline form dissolves can correspond to the amount of the drug substance that is available to be absorbed into the body to provide the intended therapeutic effect. One measure of solubility is intrinsic dissolution rate (IDR), which is defined as the dissolution rate of a substance under constant surface area conditions. For low solubility substances, higher IDR values can correlate with higher bioavailability following administration. However, if the goal is to establish bioequivalence to an existing form of a drug under investigation, such as remdesivir, substances with similar IDR values to the known form are preferred. Alternatively, for the development of extended or sustained release products, forms exhibiting lower IDR values are often preferable since they can provide slower dissolution of the drug independent of the excipients used in the formulation. Prediction of the solubility and IDR of an as yet undiscovered salt or crystalline form of a substance is currently not possible.

Different crystalline and/or salt forms of the same compound may have different crystal packing, thermodynamic, spectroscopic, kinetic, surface and mechanical properties. For example, different salts and/or crystalline forms may have different stability properties such that a particular form may be less sensitive to heat, relative humidity (RH) and/or light. Different salts and/or crystalline forms of a compound may also be more susceptible to moisture uptake, resulting in a potential alteration of physical characteristics of the form such as flowability, density or compressibility, which can lead to problems during formulation and/or to changes in dissolution rate of the formulated drug product.

For example, a particular salt and/or crystalline form may provide more favourable compressibility and/or density properties, thereby providing more desirable characteristics for formulation and/or product manufacturing. Differences in stability between salts and/or crystalline forms of a drug may result from changes in chemical reactivity, such as differential oxidation. The melting point of a particular salt and/or crystalline form, particularly a low melting point, can contribute to issues during processing, which impact on both flow and compressibility performance. Particular salts and/or crystalline forms may also have different solubilities, thereby providing different pharmacokinetic parameters, which allow for specific salts and/or crystalline forms to be used in order to achieve specific pharmacokinetic targets. Differences in solubility between salts and/or crystalline forms are particularly relevant for compounds exhibiting low solubility, such as remdesivir, where even a modest increase in solubility can provide a beneficial enhancement in bioavailability. A significant increase in the solubility or permeability of a drug such as remdesivir, which exhibits poor oral bioavailability, could be a factor in the provision of an oral formulation, for example.

Although general approaches to salt and crystalline form screening of active pharmaceutical ingredients are known, it is well established that the prediction of whether any given compound will exhibit polymorphism is not possible. Accordingly, it is not possible to extend generalities to the number and kinds of crystalline forms that can exist for remdesivir salts, or to what methods will be suitable for the preparation of any given form. Furthermore, prediction of the properties of any unknown salts and/or crystalline forms, and how they will differ from other crystalline forms or salts of the same compound, remains elusive (Joel Bernstein, Polymorphism in Molecular Crystals, Oxford University Press, New York, 2002, page 9).

Owing to the reported low solubility, poor aqueous chemical stability, and dosage form limitations associated with the known forms of remdesivir, there exists a need for novel salts and crystalline forms of remdesivir having improved properties for use in providing drug products containing remdesivir, and commercially amenable processes for their manufacture. Furthermore, the urgency surrounding the development and widespread deployment of effective treatment options for COVID-19 cannot be overstated.

### SUMMARY OF THE INVENTION

The remdesivir salts and crystalline forms of the present invention comprise pharmaceutically acceptable acids which are classified as either first class or second class acids according to a notable reference book on the pharmaceutical acceptability of salts: P. Heinrich Stahl, Camille G. Wermuth (Eds.), Handbook of Pharmaceutical Salts Properties, Selection, and Use; 2002. First class acids are classified by Stahl et al. as those that afford physiologically ubiquitous ions or metabolites in biochemical pathways, supporting their unrestricted use in pharmaceuticals whereas second class acids, whilst not naturally occurring, are used profusely and show low toxicity and good tolerability.

The remdesivir salts and crystalline forms of the present invention exhibit differences in properties when compared to the known salt and crystalline forms of remdesivir such as the maleate Form I salt reported in WO 2018/204198 A1 or the neutral remdesivir crystalline form(s) used in the VEKLURY® drug product. Depending on the specific salts and crystalline forms of the invention used, properties that may differ between the invention and known salt and crystalline forms of remdesivir include the following: packing properties such as molar volume, density and hygroscopicity, thermodynamic properties such as melting point and solubility, kinetic properties such as intrinsic dissolution rate and chemical/crystalline form stability, surface properties such as crystal habit and mechanical properties such as hardness, tensile strength, compactibility, tableting, handling, flow, and blending.

Thus, the present invention provides new salt and crystalline forms of remdesivir having advantages over known forms of remdesivir that can be exploited in the development of new formulations and dosage forms containing remdesivir.

Accordingly, in a first aspect of the present invention, there is provided a napsylate salt of remdesivir. In a preferred embodiment of the first aspect, the molar ratio of remdesivir to naphthalene-2-sulfonic acid is approximately 1:1. In a more preferred embodiment of the first aspect, the salt is characterized by a PXRD diffractogram comprising peaks, expressed in degrees 2θ (±0.2°), at 5.1°, 6.5° and 13.1°. More preferably, the salt of the first aspect is characterized by a PXRD diffractogram further comprising at least three peaks, expressed in degrees 2θ (±0.2°), selected from the group consisting of: 4.5°, 9.0°, 10.0°, 11.5°, 13.6°, 15.3°, 16.4°, 17.2°, 20.2° and 24.3°. In a further preferred embodiment of the first aspect, the PXRD diffractogram further comprises peaks, expressed in degrees 2θ (±0.21, at 4.5°, 9.0°, 10.0°, 11.5°, 13.6°, 15.3°, 16.4°, 17.2°, 20.2° and 24.3°. Preferably, the salt of the first aspect of the invention provides a PXRD diffractogram comprising peaks in substantially the same positions (±0.2°2θ) as those shown in FIG. 1. In a further preferred embodiment of the first aspect, the salt is characterized by a DSC thermogram comprising an endothermic peak with a peak onset at approximately 170° C. and a peak maximum at approximately 176° C. Preferably, the salt of the first aspect is characterized by a DSC thermogram that is substantially the same in appearance as the DSC thermogram provided in FIG. 8.

In a second aspect of the present invention, there is provided a tosylate salt of remdesivir. In a preferred embodiment of the second aspect, the molar ratio of remdesivir to p-toluenesulfonic acid is approximately 1:1. In a more preferred embodiment of the second aspect, the salt is characterized by a PXRD diffractogram comprising peaks, expressed in degrees 2θ (±0.21, at 5.5°, 7.7° and 13.1°. More preferably, the salt of the second aspect is characterized by a PXRD diffractogram further comprising at least three peaks, expressed in degrees 2θ (±0.201, selected from the group consisting of: 4.5°, 6.5°, 9.1°, 9.8°, 16.0°, 16.6°, 17.1°, 17.5°, 18.4° and 20.0°. In a further preferred embodiment of the second aspect, the PXRD diffractogram further comprises peaks, expressed in degrees 2θ (±0.2), at 4.5°, 6.5°, 9.1°, 9.8°, 16.0°, 16.6°, 17.1°, 17.5°, 18.4° and 20.0°. Preferably, the salt of the second aspect of the invention provides a PXRD diffractogram comprising peaks in substantially the same positions (±0.2° 20) as those shown in FIG. 2. In a further preferred embodiment of the second aspect, the salt is characterized by a DSC thermogram comprising an endothermic peak with a peak onset at approximately 168° C. and a peak maximum at approximately 171° C. Preferably, the salt of the second aspect is characterized by a DSC thermogram that is substantially the same in appearance as the DSC thermogram provided in FIG. 9.

In a third aspect of the present invention, there is provided a hydrochloride salt of remdesivir. In a preferred embodiment of the third aspect, the molar ratio of remdesivir to hydrochloric acid is approximately 1:1. In a more preferred embodiment of the third aspect, the salt is characterized by a PXRD diffractogram comprising peaks, expressed in degrees 2θ (±0.21, at 9.2°, 16.4° and 19.9°. More preferably, the salt of the third aspect is characterized by a PXRD diffractogram further comprising at least three peaks, expressed in degrees 2θ (±0.21, selected from the group consisting of: 13.4°, 18.3°, 20.6°, 21.6° and 24.2°. In a further preferred embodiment of the third aspect, the PXRD diffractogram further comprises peaks, expressed in degrees 2θ (±0.21, at 13.4°, 18.3°, 20.6°, 21.6° and 24.2°. Preferably, the salt of the third aspect of the invention provides a PXRD diffractogram comprising peaks in substantially the same positions (±0.2° 20) as those shown in FIG. 3.

In a fourth aspect of the present invention, there is provided a phosphate salt of remdesivir. In a more preferred embodiment of the fourth aspect, the salt is characterized by a PXRD diffractogram comprising peaks, expressed in degrees 2θ (±0.21, at 13.2°, 14.4° and 23.30. More preferably, the salt of the fourth aspect is characterized by a PXRD diffractogram further comprising at least three peaks, expressed in degrees 2θ (±0.2°), selected from the group consisting of: 11.1°, 11.5°, 16.8°, 17.6° and 30.2°. In a further preferred embodiment of the fourth aspect, the PXRD diffractogram further comprises peaks, expressed in degrees 2θ (±0.201, at 11.1°, 11.5°, 16.8°, 17.6° and 30.2°. Preferably, the salt of the fourth aspect of the invention provides a PXRD diffractogram comprising peaks in substantially the same positions (±0.2°2θ) as those shown in FIG. 4.

In a fifth aspect of the present invention, there is provided a maleate salt of remdesivir. In a more preferred embodiment of the fifth aspect, the salt is characterized by a PXRD diffractogram comprising peaks, expressed in degrees 2θ (±0.21, at 10.7°, 12.8° and 16.30. More preferably, the salt of the fifth aspect is characterized by a PXRD diffractogram further comprising at least three peaks, expressed in degrees 2θ (±0.2), selected from the group consisting of: 8.1°, 11.1°, 13.9°, 14.7°, 17.0°, 17.7°, 19.8°, 21.00, 22.6° and 24.5°. In a further preferred embodiment of the fifth aspect, the PXRD diffractogram further comprises peaks, expressed in degrees 2θ (±0.2), at 8.1°, 11.1°, 13.90, 14.7°, 17.0°, 17.7°, 19.8°, 21.0°, 22.6° and 24.5°. Preferably, the salt of the fifth aspect of the invention provides a PXRD diffractogram comprising peaks in substantially the same positions (±0.2° 2θ) as those shown in FIG. 5.

In a sixth aspect of the present invention, there is provided an oxalate salt of remdesivir. In a first preferred embodiment of the sixth aspect, the salt is characterized by a PXRD diffractogram comprising peaks, expressed in degrees 2θ (±0.201, at 7.4°, 10.3° and 22.9°. Preferably, the salt of this embodiment is characterized by a PXRD diffractogram further comprising at least three peaks, expressed in degrees 2θ (±0.21, selected from the group consisting of: 9.7°, 11.4°, 12.1°, 17.1°, 18.6°, 20.2° and 21.7°. More preferably, the PXRD diffractogram of this embodiment further comprises peaks, expressed in degrees 2θ (±0.20), at 9.7°, 11.4°, 12.1°, 17.1°, 18.6°, 20.2° and 21.7°. Preferably in this embodiment, the salt provides a PXRD diffractogram comprising peaks in substantially the same positions (±0.2°2θ) as those shown in FIG. 6. In a second preferred embodiment of the sixth aspect, the salt is characterized by a PXRD diffractogram comprising peaks, expressed in degrees 2θ (±0.2θ), at 5.3°, 7.2° and 16.4°. Preferably, the salt of this embodiment is characterized by a PXRD diffractogram further comprising at least three peaks, expressed in degrees 2 (±0.20), selected from the group consisting of: 8.2°, 8.6°, 11.5°, 14.3° and 19.8°. More preferably, the PXRD diffractogram of this embodiment further comprises peaks, expressed in degrees 2θ (±0.2), at 8.2°, 8.6°, 11.5°, 14.3° and 19.80. Preferably in this embodiment, the salt provides a PXRD diffractogram comprising peaks in substantially the same positions (±0.2°2θ) as those shown in FIG. 7.

In a seventh aspect of the present invention, there is provided a pharmaceutical composition comprising a salt of remdesivir according to the first, second, third, fourth, fifth, or sixth aspects of the invention, and one or more pharmaceutically acceptable excipients. Preferably, the pharmaceutical composition is in the form of a lyophilized composition or a solution composition. Preferably, the pharmaceutical composition of the seventh aspect comprises an amount of the remdesivir salt of the first, second, third, fourth, fifth or sixth aspects that is equivalent to 100 mg of remdesivir.

In an eighth aspect of the present invention, there is provided the use of a salt of remdesivir according to the first, second, third, fourth, fifth, or sixth aspects of the invention, or the pharmaceutical compositions of the seventh aspect of the invention, in the treatment of a viral infection. In a preferred embodiment of the eighth aspect, the viral infection is caused by a virus selected from the group consisting of an Arenavirdae virus, a Coronaviridae virus, a Filoviridae virus, a Flaviviridae virus, and a Paramyxoviridae virus. In a further preferred embodiment of the eighth aspect, the viral infection is caused by a virus selected from the group consisting of Lassa virus, Junin virus, Severe Acute Respiratory Syndrome coronavirus (SARS-CoV), Severe Acute Respiratory Syndrome coronavirus 2 (SARS-CoV-2), Middle Eastern Respiratory Syndrome coronavirus (MERS-CoV), Ebola virus, Marburg virus, Zika virus, and Respiratory Syncytial virus (RSV). In a more preferred embodiment of the eighth aspect, the viral infection is caused by Severe Acute Respiratory Syndrome coronavirus 2 (SARS-CoV-2).

In a ninth aspect of the present invention, there is provided a method of treating a viral infection comprising administering a therapeutically effective amount of the salt of remdesivir according to the first, second, third, fourth, fifth, or sixth aspects of the invention, or the pharmaceutical compositions of the seventh aspect of the invention, to a patient in need thereof. In a preferred embodiment of the ninth aspect, the viral infection is caused by a virus selected from the group consisting of an Arenaviridae virus, a Coronaviridae virus, a Filoviridae virus, a Flaviviridae virus, and a Paramyxoviridae virus. In a further preferred embodiment of the ninth aspect, the viral infection is caused by a virus selected from the group consisting of Lassa virus, Junin virus, Severe Acute Respiratory Syndrome coronavirus (SARS-CoV), Severe Acute Respiratory Syndrome coronavirus 2 (SARS-CoV-2), Middle Eastern Respiratory Syndrome coronavirus (MERS-CoV), Ebola virus, Marburg virus, Zika virus, and Respiratory Syncytial virus (RSV). In a more preferred embodiment of the ninth aspect, the viral infection is caused by Severe Acute Respiratory Syndrome coronavirus 2 (SARS-CoV-2).

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention are described, by way of example only, with reference to the attached Figures.

FIG. 1 is a representative PXRD diffractogram of remdesivir napsylate Form APO-I as prepared in Example 1.

FIG. 2 is a representative PXRD diffractogram of remdesivir tosylate Form APO-I as prepared in Example 2.

FIG. 3 is a representative PXRD diffractogram of remdesivir hydrochloride Form APO-I as prepared in Example 3.

FIG. 4 is a representative PXRD diffractogram of remdesivir phosphate Form APO-I as prepared in Example 4.

FIG. 5 is a representative PXRD diffractogram of remdesivir maleate Form APO-I as prepared in Example 5.

FIG. 6 is a representative PXRD diffractogram of remdesivir oxalate Form APO-I as prepared in Example 6.

FIG. 7 is a representative PXRD diffractogram of remdesivir oxalate Form APO-II as prepared in Example 7.

FIG. 8 is a representative DSC thermogram of remdesivir napsylate Form APO-I as prepared in Example 1.

FIG. 9 is a representative DSC thermogram of remdesivir tosylate Form APO-1 as prepared in Example 2.

### DESCRIPTION OF THE INVENTION

The present invention provides novel salts of remdesivir and crystalline forms thereof providing improved properties over known salts and/or crystalline forms of remdesivir.

The remdesivir salts and crystalline forms of the present invention exhibit differences in properties when compared to known salts and/or crystalline forms of remdesivir. Depending on the specific salts and crystalline forms of the invention used, properties that differ between the invention and known salts and crystalline forms of remdesivir include crystal packing properties such as molar volume, density and hygroscopicity; thermodynamic properties such as melting point and solubility; kinetic properties such as dissolution rate and chemical/polymorphic stability; surface properties such as crystal habit/particle morphology; and/or mechanical properties such as hardness, tensile strength, compactibility, tabletting, handling, flow, and blending. The improved properties provided by the salts and crystalline forms of the present invention provide practical advantages over known forms of remdesivir that can be exploited to meet specific needs in the manufacture and formulation of remdesivir.

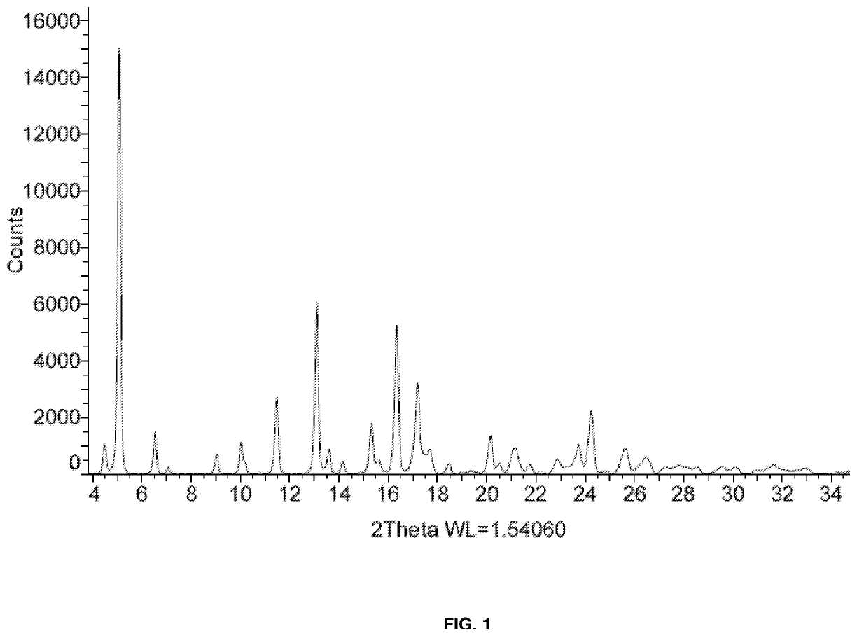

Depending on the manner in which the crystalline forms of the present invention are prepared, and the methodology and instrument used for PXRD analysis, the intensity of a given peak observed in a PXRD diffractogram of a crystalline form may vary when compared to the same peak in the representative PXRD diffractograms provided in FIGS. 1 to 7. Thus, differences in relative peak intensities between peaks in a PXRD diffractogram for a given crystalline form may be observed when compared to the relative peak intensities of the peaks in the representative PXRD diffractograms of FIGS. 1 to 7. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractograms provided in FIGS. 1 to 7, it is understood that individual peak positions may vary between ±0.2° 2θ from the values observed in the representative PXRD diffractograms provided in FIGS. 1 to 7 for the crystalline form of the invention, or listed in Tables 1 to 7. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 7. Thus, PXRD diffractograms of the crystalline form of the present invention may, in some circumstances, display the same relative peak positions as observed in the representative PXRD diffractograms provided in FIGS. 1 to 7, with the exception that each peak is offset in the same direction, and by approximately the same amount, such that the overall PXRD diffractogram is substantially the same in appearance as the PXRD diffractograms of FIGS. 1 to 7, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractograms of FIGS. 1 to 7.

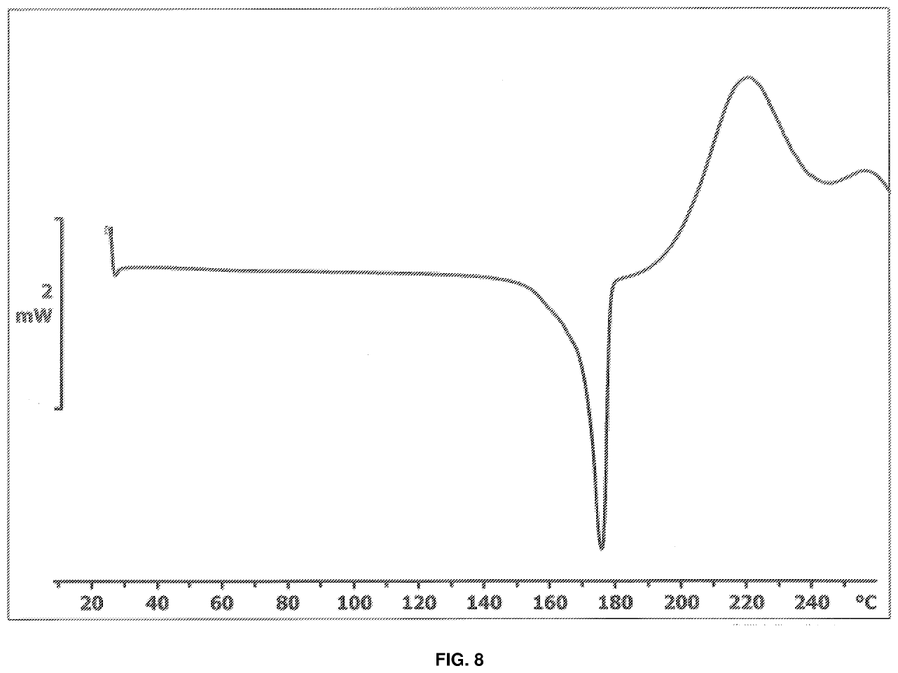

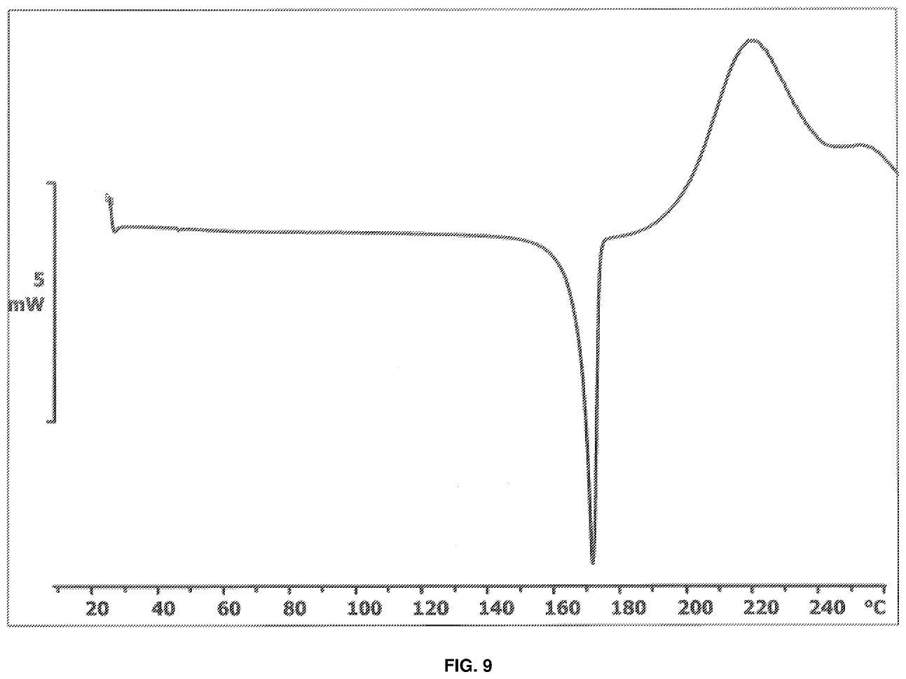

Depending on the manner in which the crystalline forms are prepared, the methodology and instrument used for DSC analysis, it is understood that peaks corresponding with thermal events in a DSC thermogram may vary between ±2° C. from the values observed in the representative DSC thermograms provided in FIGS. 8 and 9 and described herein. Such variations are known and understood by a person of skill in the art, and any such variations do not depart from the invention disclosed herein.

As used herein, the term ‘crystalline form’ refers to a substance with a particular arrangement of molecular components in its crystal lattice, and which may be identified by physical characterization methods such as PXRD and/or DSC.

As used herein, the term “room temperature” refers to a temperature in the range of 20° C. to 25° C.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In a first embodiment of the present invention, there is provided a new salt of remdesivir, remdesivir napsylate Form APO-I, wherein the molar ratio of remdesivir to naphthalene-2-sulfonic acid is approximately 1:1.

Remdesivir napsylate Form APO-I can be characterized by a PXRD diffractogram comprising, among other peaks, characteristic peaks, expressed in degrees 2θ (±0.2°), at 5.1°, 6.5° and 13.1°. Preferably, the PXRD diffractogram further comprises at least three peaks, expressed in degrees 2θ (±0.2°), selected from the group consisting of 4.5°, 9.0°, 10.0°, 11.5°, 13.6°, 15.3°, 16.4°, 17.2°, 20.2° and 24.3°. More preferably, the PXRD diffractogram further comprises peaks, expressed in degrees 2θ (±0.2°), at 4.5°, 9.0°, 10.0°, 11.5°, 13.6°, 15.3°, 16.4°, 17.2°, 20.2° and 24.3°. PXRD studies of capped and uncapped samples of remdesivir napsylate Form APO-I maintained in a 40° C./75% RH stability chamber for at least one year showed that no change in the crystalline form occurred.

An illustrative PXRD diffractogram of remdesivir napsylate Form APO-I, as prepared in Example 1, is shown in FIG. 1. A peak listing, comprising representative peaks from the PXRD diffractogram in FIG. 1, and their relative intensities, is provided in Table 1. Although illustrative of the PXRD diffractogram that is provided for the remdesivir napsylate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of remdesivir napsylate
Form APO-I from FIG. 1

| | Relative |
|---|---|
| | intensity |
| Angle (2θ) | (%) |
| 4.48 | 6.9 |
| 5.08 | 100.0 |
| 6.53 | 9.9 |
| 9.04 | 4.7 |
| 10.03 | 7.2 |
| 11.48 | 18.0 |
| 13.10 | 40.3 |
| 13.60 | 5.8 |
| 15.33 | 11.9 |
| 16.36 | 35.0 |
| 17.20 | 21.3 |
| 20.16 | 8.9 |
| 21.15 | 6.2 |
| 24.25 | 15.1 |

An illustrative DSC thermogram of remdesivir napsylate Form APO-I is shown in FIG. 8. The DSC thermogram may be further characterized by an endothermic peak with a peak onset at approximately 170° C. and a peak maximum at approximately 176° C.

In a second embodiment of the present invention, there is provided a new salt of remdesivir, remdesivir tosylate Form APO-I, wherein the molar ratio of remdesivr to p-toluenesulfonic acid is approximately 1:1.

Remdesivir tosylate Form APO-I can be characterized by a PXRD diffractogram comprising, among other peaks, characteristic peaks, expressed in degrees 28 (±0.2°), at 5.5°, 7.7° and 13.1°. Preferably, the PXRD diffractogram further comprises at least three peaks, expressed in degrees 2θ (±0.2°), selected from the group consisting of 4.5°, 6.5°, 9.1°, 9.8°, 16.0°, 16.6°, 17.1°, 17.5°, 18.4° and 20.0°. More preferably, the PXRD diffractogram further comprises peaks, expressed in degrees 2θ (±0.2°), at 4.5°, 6.5°, 9.1°, 9.8°, 16.0°, 16.6°, 17.1°, 17.5°, 18.4° and 20.0°. PXRD studies of capped and uncapped samples of remdesivir tosylate Form APO-1 maintained in a 40° C./75% RH stability chamber for at least one year showed that no change in the crystalline form occurred

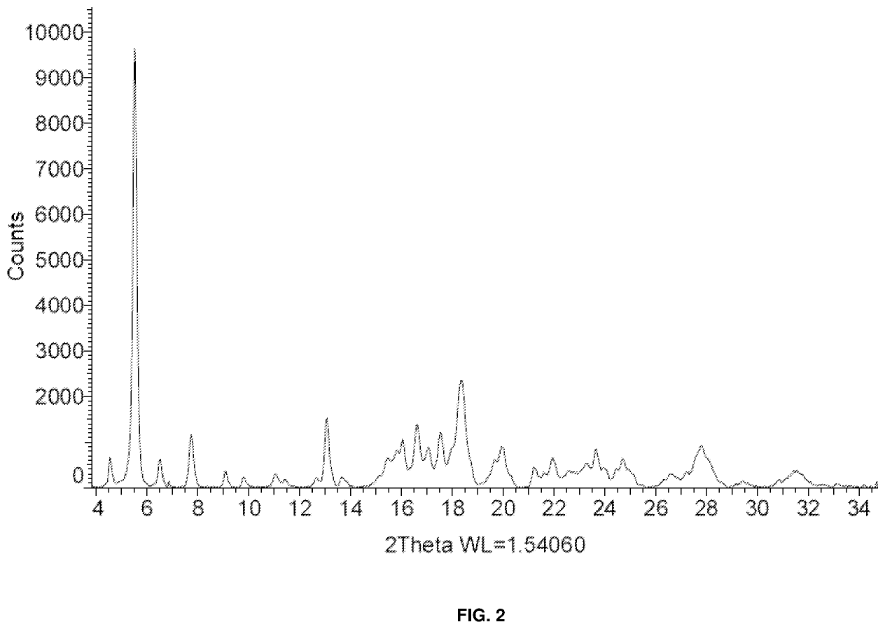

An illustrative PXRD diffractogram of remdesivir tosylate Form APO-I, as prepared in Example 2, is shown in FIG. 2. A peak listing, comprising representative peaks from the PXRD diffractogram in FIG. 2, and their relative intensities, is provided in Table 2. Although illustrative of the PXRD diffractogram that is provided for the remdesivir tosylate Form APO-1 of the present invention, the relative intensities of the peaks are variable. Thus, depending on a particular sample, the prominence or relative intensity of the peaks observed may differ from those in the illustrative PXRD diffractogram and peak listing.

TABLE 2
Relative peak intensities of remdesivir tosylate
Form APO-I from FIG. 2

| | Relative |
|---|---|
| | intensity |
| Angle (2θ) | (%) |
| 4.54 | 6.8 |
| 5.51 | 100.0 |
| 6.51 | 6.5 |
| 7.73 | 11.9 |
| 9.09 | 3.6 |
| 9.79 | 2.4 |
| 13.06 | 16.0 |
| 15.46 | 6.8 |
| 15.84 | 8.5 |
| 16.04 | 10.9 |
| 16.63 | 14.3 |
| 17.05 | 9.2 |
| 17.55 | 12.6 |
| 18.37 | 24.4 |
| 19.96 | 9.3 |
| 21.95 | 6.8 |

An illustrative DSC thermogram of remdesivir tosylate Form APO-I is shown in FIG. 9. The DSC thermogram may be further characterized by an endothermic peak with a peak onset at approximately 168° C. and a peak maximum at approximately 171° C.

In a third embodiment of the present invention, there is provided a new salt of remdesivir, remdesivir hydrochloride Form APO-I, wherein the molar ratio of remdesivir to hydrochloric acid is approximately 1:1.

Remdesivir hydrochloride Form APO-I can be characterized by a PXRD diffractogram comprising, among other peaks, characteristic peaks, expressed in degrees 2θ (±0.2°), at 9.20, 16.4° and 19.9°. Preferably, the PXRD diffractogram further comprises at least three peaks, expressed in degrees 2θ (±0.2°), selected from the group consisting of 13.4°, 18.3°, 20.6°, 21.6° and 24.2°. More preferably, the PXRD diffractogram further comprises peaks, expressed in degrees 2θ (±0.2°), at 13.4°, 18.3°, 20.6°, 21.6° and 24.2°.

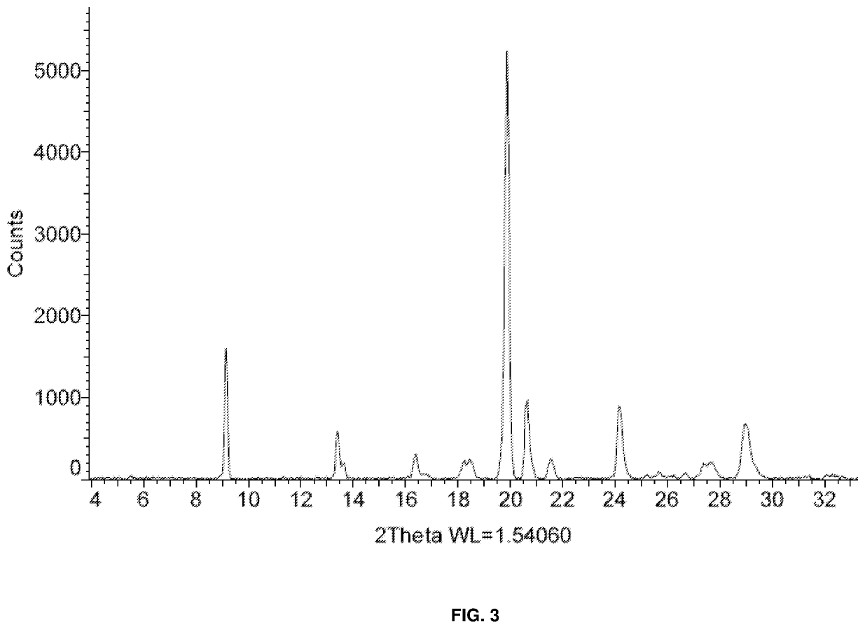

An illustrative PXRD diffractogram of remdesivir hydrochloride Form APO-I, as prepared in Example 3, is shown in FIG. 3. A peak listing, comprising representative peaks from the PXRD diffractogram in FIG. 3, and their relative intensities, is provided in Table 3. Although illustrative of the PXRD diffractogram that is provided for the remdesivir hydrochloride Form APO-I of the present invention, the relative intensities of the peaks are variable. Thus, depending on a particular sample, the prominence or relative intensity of the peaks observed may differ from those in the illustrative PXRD diffractogram and peak listing.

TABLE 3
Relative peak intensities of remdesivir hydrochloride
Form APO-I from FIG. 3

| | Relative |
|---|---|
| | intensity |
| Angle (2θ) | (%) |
| 9.15 | 30.3 |
| 13.40 | 11.1 |
| 16.40 | 5.8 |
| 18.26 | 4.4 |
| 19.87 | 100.0 |
| 20.64 | 18.1 |
| 21.56 | 4.8 |
| 24.17 | 17.0 |
| 28.99 | 12.7 |

In a fourth embodiment of the present invention, there is provided a new salt of remdesivir, remdesivir phosphate Form APO-I, comprising remdesivir and phosphoric acid.

Remdesivir phosphate Form APO-I can be characterized by a PXRD diffractogram comprising, among other peaks, characteristic peaks, expressed in degrees 2θ (±0.2°), at 13.2°, 14.4° and 23.30. Preferably, the PXRD diffractogram further comprises at least three peaks, expressed in degrees 2θ (±0.2°), selected from the group consisting of 11.1°, 11.5°, 16.8°, 17.6° and 30.2°. More preferably, the PXRD diffractogram further comprises peaks, expressed in degrees 2θ (±0.2°), at 11.1°, 11.5°, 16.8°, 17.6° and 30.2°.

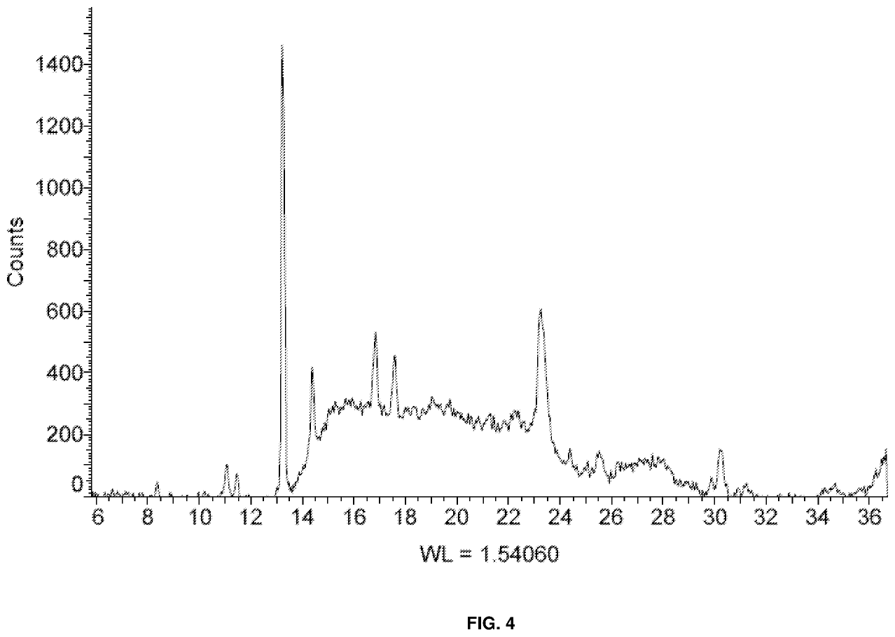

An illustrative PXRD diffractogram of remdesivir phosphate Form APO-I, as prepared in Example 4, is shown in FIG. 4. A peak listing, comprising representative peaks from the PXRD diffractogram in FIG. 4, and their relative intensities, is provided in Table 4. Although illustrative of the PXRD diffractogram that is provided for the remdesivir phosphate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 4
Relative peak intensities of remdesivir phosphate
Form APO-I from FIG. 4

| | Relative |
|---|---|
| | intensity |
| Angle (2θ) | (%) |
| 11.07 | 7.0 |
| 11.47 | 4.8 |
| 13.22 | 100.0 |
| 14.39 | 28.4 |
| 16.84 | 36.4 |
| 17.59 | 31.1 |
| 23.25 | 41.2 |
| 30.21 | 10.3 |

In a fifth embodiment of the present invention, there is provided a new salt of remdesivir, remdesivir maleate Form APO-I, comprising remdesivir and maleic acid.

Remdesivir maleate Form APO-I can be characterized by a PXRD diffractogram comprising, among other peaks, characteristic peaks, expressed in degrees 2θ (±0.2°), at 10.7°, 12.8° and 16.30. Preferably, the PXRD diffractogram further comprises at least three peaks, expressed in degrees 2θ (±0.2°), selected from the group consisting of 8.1°, 11.1°, 13.9°, 14.7°, 17.0°, 17.7°. 19.8°, 21.0°, 22.6° and 24.5°. More preferably, the PXRD diffractogram further comprises peaks, expressed in degrees 2θ (±0.2), at 8.1°, 11.1°, 13.9°, 14.7°, 17.0°, 17.7°. 19.8°, 21.0°, 22.6° and 24.5°.

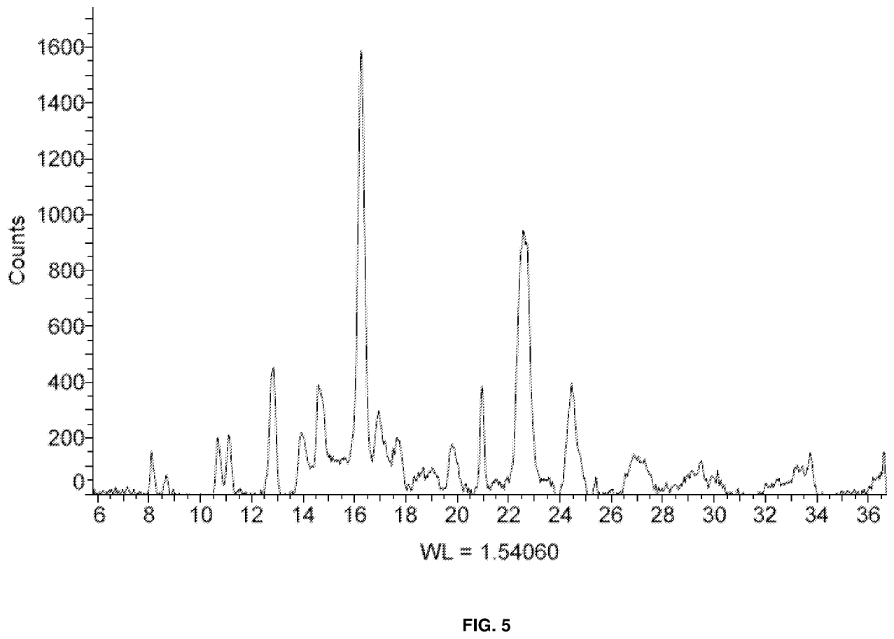

An illustrative PXRD diffractogram of remdesivir maleate Form APO-I, as prepared in Example 5, is shown in FIG. 5. A peak listing, comprising representative peaks from the PXRD diffractogram in FIG. 5, and their relative intensities, is provided in Table 5. Although illustrative of the PXRD diffractogram that is provided for the remdesivir maleate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 5
Relative peak intensities of remdesivir maleate
Form APO-I from FIG. 5

| | Relative |
|---|---|
| | intensity |
| Angle (2θ) | (%) |
| 8.11 | 9.6 |
| 8.68 | 4.2 |
| 10.69 | 12.7 |
| 11.11 | 13.2 |
| 12.84 | 28.5 |
| 13.95 | 13.8 |
| 14.65 | 23.7 |
| 16.26 | 100.0 |
| 16.95 | 18.7 |
| 17.69 | 12.1 |
| 19.80 | 11.3 |
| 20.96 | 24.2 |
| 22.58 | 59.4 |
| 24.46 | 25.0 |

In a sixth embodiment of the present invention, there is provided a new salt of remdesivir, remdesivir oxalate Form APO-I, comprising remdesivir and oxalic acid.

Remdesivir oxalate Form APO-I can be characterized by a PXRD diffractogram comprising, among other peaks, characteristic peaks, expressed in degrees 2θ (±0.2°), at 7.4°, 10.3° and 22.9°. Preferably, the PXRD diffractogram further comprises at least three peaks, expressed in degrees 2θ (±0.2°), selected from the group consisting of 9.7°, 11.40, 12.1°, 17.1°, 18.6°, 20.2° and 21.7°. More preferably, the PXRD diffractogram further comprises peaks, expressed in degrees 2θ (±0.2°), at 9.7°, 11.4°, 12.1°, 17.10, 18.6°, 20.2° and 21.7°.

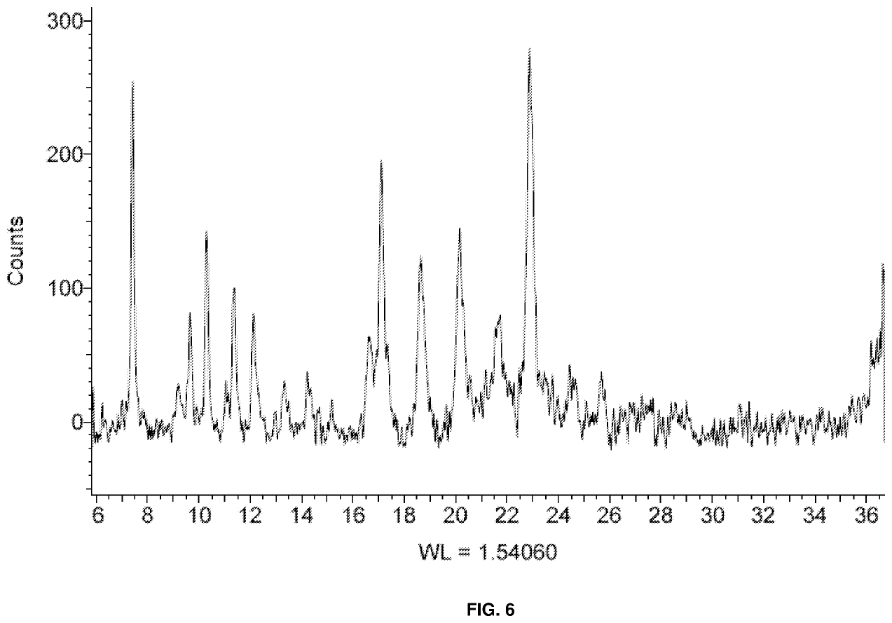

An illustrative PXRD diffractogram of remdesivir oxalate Form APO-I, as prepared in Example 6, is shown in FIG. 6. A peak listing, comprising representative peaks from the PXRD in FIG. 6, and their relative intensities, is provided in Table 6. Although illustrative of the PXRD diffractogram that is provided for the remdesivir oxalate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 6
Relative peak intensities of remdesivir oxalate
Form APO-I from FIG. 6

| | Relative |
|---|---|
| | intensity |
| Angle (2θ) | (%) |
| 7.41 | 90.4 |
| 9.66 | 29.2 |
| 10.31 | 50.5 |
| 11.38 | 35.6 |
| 12.13 | 28.7 |
| 16.68 | 21.5 |
| 17.10 | 69.8 |
| 18.64 | 44.2 |
| 20.16 | 51.6 |
| 21.67 | 26.8 |
| 22.88 | 100.0 |

In a seventh embodiment of the present invention, there is provided a new salt of remdesivir, remdesivir oxalate Form APO-II, comprising remdesivir and oxalic acid.

Remdesivir oxalate Form APO-II can be characterized by a PXRD diffractogram comprising, among other peaks, characteristic peaks, expressed in degrees 2θ (±0.2°), at 5.3°, 7.2° and 16.4°. Preferably, the PXRD diffractogram further comprises at least three peaks, expressed in degrees 2θ (±0.2°), selected from the group consisting of 8.2°, 8.6°, 11.5°, 14.3° and 19.8°. More preferably, the PXRD diffractogram further comprises peaks, expressed in degrees 2θ (±0.2°), at 8.2°, 8.6°, 11.5°, 14.3° and 19.8°.

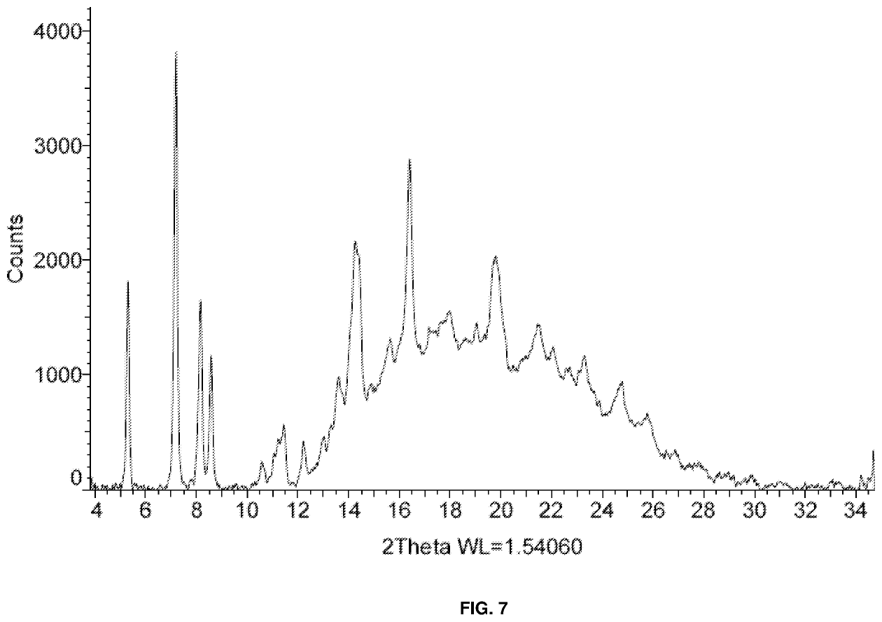

An illustrative PXRD diffractogram of remdesivir oxalate Form APO-II, as prepared in Example 7, is shown in FIG. 7. A peak listing, comprising representative peaks from the PXRD in FIG. 7, and their relative intensities, is provided in Table 7. Although illustrative of the PXRD diffractogram that is provided for the remdesivir oxalate Form APO-II of the present invention, the relative intensities of the peaks are variable. Thus, depending on a particular sample, the prominence or relative intensity of the peaks observed may differ from those in the illustrative PXRD diffractogram and peak listing.

TABLE 7
Relative peak intensities of remdesivir oxalate
Form APO-II from FIG. 7

| | Relative |
|---|---|
| | intensity |
| Angle (2θ) | (%) |
| 5.30 | 47.8 |
| 7.19 | 100.0 |
| 8.17 | 43.5 |
| 8.58 | 30.9 |
| 11.45 | 14.8 |
| 12.22 | 11.0 |
| 13.62 | 25.9 |
| 14.32 | 55.3 |
| 16.41 | 75.8 |
| 19.81 | 53.7 |
| 21.46 | 38.1 |
| 23.31 | 30.7 |

In an eighth embodiment of the invention, there is provided a pharmaceutical composition comprising one or more remdesivir salt(s) selected from the group consisting of remdesivir napsylate, remdesivir tosylate, remdesivir hydrochloride, remdesivir phosphate, remdesivir maleate, remdesivir oxalate, and combinations thereof, with one or more pharmaceutically acceptable excipients. Preferably, the pharmaceutical composition comprises one or more crystalline form(s) of a remdesivir salt selected from the group consisting of remdesivir napsylate Form APO-I, remdesivir tosylate Form APO-I, remdesivir hydrochloride Form APO-I, remdesivir phosphate Form APO-I, remdesivir maleate Form APO-I, remdesivir oxalate Form APO-I, remdesivir oxalate Form APO-II and combinations thereof. Preferably, the pharmaceutical composition is a dosage form suitable for parenteral or inhalation administration, such as a lyophilized formulation or a solution formulation. Most preferably, the pharmaceutical composition is a powder for concentrate for solution for infusion or a concentrate for solution for infusion.

As used herein, the phrase “therapeutically effective amount” means that amount of remdesivir salt (salt form of remdesivir comprising remdesivir and counter-ion), or crystalline form thereof, that will elicit a biological or medical response of a tissue, system, or patient that is being sought by the administrator (such as a researcher, doctor, or veterinarian) which includes alleviation of the symptoms of the condition or disease being treated and the prevention, slowing, or halting of progression of the condition or disease, including but not limited to viral infection. In some examples, the pharmaceutical preparation is in a unit dosage form. In such a form, the preparation is subdivided into suitably sized unit doses containing appropriate quantities of the active component, e.g., an effective amount to achieve the desired purpose.

The quantity of remdesivir napsylate, remdesivir tosylate, remdesivir hydrochloride, remdesivir phosphate, remdesivir maleate, and/or remdesivir oxalate, in a unit dose of preparation comprises or consists of an amount of remdesivir that is equivalent to about 1 mg to about 1000 mg, or about 5 mg to about 500 mg, or about 50 mg to about 250 mg, or about 60 mg to about 240 mg, or about 70 mg to about 230 mg, or about 80 mg to about 220 mg, or about 90 mg to about 210 mg, or about 100 mg to about 200 mg, or about 90 mg to about 110 mg, or about 145 mg to about 165 mg, or about 90 mg to about 175 mg, or about 10 mg, or about 20 mg, or about 30 mg, or about 40 mg, or about 50 mg, or about 60 mg, or about 70 mg, or about 80 mg, or about 90 mg, or about 100 mg, or about 110 mg, or about 120 mg, or about 130 mg, or about 140 mg, or about 150 mg, or about 160 mg, or about 170 mg, or about 180 mg, or about 190 mg, or about 200 mg, or about 210 mg, or about 220 mg, or about 230 mg, or about 240 mg, or about 250 mg, as desired. In some examples, the mixture comprises about 90 mg to about 175 mg, or about 100 mg, or about 150 mg, of remdesivir. For example, an amount of 269 mg of remdesivir napsylate provides 200 mg of remdesivir. Similarly, an amount of 135 mg of remdesivir napsylate provides 100 mg of remdesivir. The actual dosage employed may be varied depending upon the requirements of the patient and the severity of the condition being treated.

For convenience, the total daily dosage may be divided and administered in portions during the day as required. In some examples, the individual portions can be of the same or different amounts of a given remdesivir salt or crystalline form of the present invention. In some examples, 200 mg/day of remdesivir (269 mg of remdesivir napsylate) can be administered at day ‘0’ (or sometimes referred to as day ‘1’), followed by 100 mg/day of remdesivir (135 mg of remdesivir napsylate) to a total of 5 days or a total of 10 days. In some examples, the pharmaceutical composition provides a dose of a remdesivir salt selected from the group consisting of remdesivir napsylate, remdesivir tosylate, remdesivir hydrochloride, remdesivir phosphate, remdesivir maleate, and remdesivir oxalate that is equivalent to the 100 mg or 200 mg of remdesivir found in VEKLURY® drug products. Thus, for example, a preferred composition may comprise 135 mg or 269 mg of remdesivir napsylate providing 100 mg or 200 mg remdesivir, respectively. In some examples, the dosage can range from about 0.001 to about 100 mg/kg of body weight/day of remdesivir napsylate, remdesivir tosylate, remdesivir hydrochloride, remdesivir phosphate, remdesivir maleate, and/or remdesivir oxalate, or about 0.01 to about 10 mg/kg of body weight/day. It should be understood that any numerical range recited herein is intended to include all sub-ranges subsumed therein. For example, a range of “1 to 10” is intended to include all sub-ranges between and including the recited minimum value of 1 and the recited maximum value of 10, that is, having a minimum value equal to or greater than 1 and a maximum value of equal to or less than 10.

Suitable pharmaceutically acceptable excipients for use in parenteral compositions are preferably inert with respect to the remdesivir salts and crystalline forms of the present invention, and may include, for example, one or more components selected from vehicles such as water, ethyl alcohol, liquid polyethylene glycol, and propylene glycol; fixed oils such as corn oil, cottonseed oil, peanut oil, and sesame oil; complexing agents such as cyclodextrins and Betadex sulfobutyl ether sodium; surface active agents such as polyoxyethylene sorbitan monolaurate (Tween 20) and polyoxyethylene sorbitan monooleate (Tween 80); tonicity adjusters such as sodium chloride, dextrose, and glycerin; antioxidants such as sodium bisulfite, sulfurous acids, ascorbic acid, and ethylenediaminetetraacetic acid (EDTA); chelating agents; buffers such as citrates, acetates, and phosphates; cryoprotectants and lyoprotectants such as sucrose, trehalose, glycine, lysine, polyethylene glycol, dextran, mannitol, and sorbitol. Other suitable excipients and carriers and the preparation of dosage forms is well known to person of skill in the art, and is described generally, for example, in Remington The Science and Practice of Pharmacy 21^{st} Edition (Lippincott Williams & Wilkins: Philadelphia; 2006; Chapter 41).

Alternatively, the remdesivir salts and crystalline forms of the present invention may be formulated as inhalable compositions as described in, for example, WO 2012/012776 A1, which is hereby incorporated by reference.

### Examples

The following non-limiting examples are illustrative of some of the aspects and embodiments of the invention described herein.

The remdesivir used as a starting material in the following examples was consistent with Form IV remdesivir which is reported in WO 2018/204198 A1. However, other polymorphic forms are equally suitable as starting material when preparing the novel salt and crystalline forms of remdesivir of the present invention.

### PXRD Analysis:

The PXRD diffractogram was recorded on a Bruker D8 Discover powder X-ray diffractometer (Bruker-AXS, Karlsruhe, Germany). The generator was a Micro-focus X-ray source (Incoatec IμS Cu anode, λ=1.54060 Å) with a voltage of 50 kV and current of 1.00 mA. X-rays were focused with a micro mask 0.1 mm plug-in microslit. One frame was collected using a still scan with a PILATUS3 R 100K-A detector at the distance of 154.72 mm from the sample. Raw data was evaluated using the program EVA (Bruker-AXS, Karlsruhe, Germany).

### Differential Scanning Calorimetry Analysis:

The DSC thermogram was collected on a Mettler-Toledo 821e instrument. The sample (2.5018 mg) was weighed into a 40 μL aluminum pan and was crimped closed with an aluminum lid having a 50 μm perforation. The sample was analyzed under a flow of nitrogen (50±5 mL/min) at a scan rate of 10° C./minute between 25° C. and 320° C.

### Temperature Cycling Program:

Temperature cycling in Examples 1, 2 and 6 was conducted as follows: 50° C., 2 h; 40° C., 1 h; 30° C., 1 h; 20° C., 1 h; 10° C., 1 h; 5° C., 1 h; 40° C., 1 h; 30° C., 1 h; 20° C., 1 h; 10° C., 1 h; 5° C., 1 h; 30° C., 1 h; 20° C., 1 h; 10° C., 1 h; 5° C., 1 h.

### Example 1: Preparation of Remdesivir Napsylate Form APO-I

In a sealed vial, remdesivir free base (54 mg) and naphthalene-2-sulfonic acid hydrate (23 mg) were dissolved in ethyl acetate (5.0 mL), followed by addition of tert-butyl methyl ether (10.0 mL). The resulting suspension was temperature cycled for 16 hours, after which the precipitated solid was collected by filtration, washed with tert-butyl methyl ether (2 mL) and dried in vacuo at room temperature for approximately 24 hours to afford remdesivir naphthalene-2-sulfonate Form APO-I (67 mg) as a white solid. ¹H NMR analysis of the solid (DMSO-d₆, 400 MHz) indicated a molar ratio of remdesivir:naphthalene-2-sulfonic acid of approximately 1:1. The PXRD diffractogram and DSC thermogram of a sample prepared by this method are shown in FIG. 1 and FIG. 8, respectively.

¹H NMR (DMSO-d₆, 400 MHz): 9.11 (br s, 1H), 8.65 (br s, 1H), 8.14 (s, 1H), 8.13 (s, 1H), 7.95-8.00 (m, 1H), 7.88-7.92 (m, 1H), 7.86 (d, J=8.5 Hz, 1H), 7.71 (d, J=8.3 Hz, 1H), 7.51-7.54 (m, 2H), 7.35 (t, J=7.8 Hz, 2H) 7.16-7.20 (m, 4H), 6.94 (d, J=4.5 Hz, 1H), 6.04 (dd, J=12.6, 10.4 Hz, 1H), 4.59 (d, J=4.6 Hz, 1H), 4.21-4.28 (m, 2H), 4.09 (quint, J=5.9 Hz, 1H), 3.92-3.99 (m, 2H), 3.88 (dd, J=11.0, 5.8 Hz, 1H), 3.76-3.85 (m, 1H), 1.42 (sep, J=6.1 Hz, 1H), 1.20-1.29 (m, 7H), 0.80 (t, J=7.4 Hz, 6H).

### Example 2: Preparation of Remdesivir Tosylate Form APO-I

In a sealed vial, remdesivir free base (54 mg) and p-toluenesulfonic acid monohydrate (19 mg) were dissolved in ethyl acetate (5.0 mL), followed by addition of tert-butyl methyl ether (10.0 mL). The resulting suspension was temperature cycled for 16 hours, after which the precipitated solid was collected by filtration, washed with tert-butyl methyl ether (2 mL), and dried in vacuo at room temperature for approximately 24 hours to afford remdesivir tosylate Form APO-I (58 mg) as a white solid. ¹H NMR analysis of the solid (DMSO-d₆, 400 MHz) indicated a molar ratio of remdesivir:p-toluenesulfonic acid of approximately 1:1. The PXRD diffractogram and DSC thermogram of a sample prepared by this method are shown in FIG. 2 and FIG. 9, respectively.

¹H NMR (DMSO-d₆, 400 MHz): 9.09 (br s, 1H), 8.64 (br s, 1H), 8.12 (s, 1H), 7.48 (d, J=7.9 Hz, 2H), 7.35 (t, J=7.9 Hz, 2H) 7.15-7.20 (m, 4H), 7.11 (d, J=7.9 Hz, 2H), 6.93 (d, J=4.6 Hz, 1H), 6.04 (dd, J=12.7, 10.2 Hz, 1H), 4.59 (d, J=4.8 Hz, 1H), 4.22-4.28 (m, 2H), 4.09 (quint, J=5.9 Hz, 1H), 3.92-3.99 (m, 2H), 3.88 (dd, J=11.1, 5.8 Hz, 1H), 3.76-3.85 (m, 1H), 2.29 (s, 3H), 1.42 (sep, J=6.2 Hz, 1H), 1.20-1.29 (m, 7H), 0.80 (t, J=7.4 Hz, 6H).

### Example 3: Preparation of Remdesivir Hydrochloride Form APO-1

To remdesivir free base (5 mg) in methyl isobutyl ketone or n-butanol (200 μL) was added a solution of concentrated HCl (3.3 μL) in ethanol (50 μL). The clear solution was covered with a tissue and set aside to allow the solvent to slowly evaporate over seven days. The PXRD diffractogram of the resulting crystals of remdesivir hydrochloride Form APO-1 is shown in FIG. 3.

### Example 4: Preparation of Remdesivir Phosphate Form APO-1

To remdesivir free base (5 mg) in methanol (200 μL) was added a solution of 85% aqueous H₃PO₄ (7.7 μL) in methanol (50 μL). The clear solution was covered with a tissue and set aside to allow the solvent to slowly evaporate over four days. The PXRD diffractogram of the resulting crystals of remdesivir phosphate Form APO-1 is shown in FIG. 4.

### Example 5: Preparation of Remdesivir Maleate Form APO-1

To remdesivir free base (5 mg) in methanol (250 μL) was added maleic acid (1.04 mg). The clear solution was covered with a tissue and set aside to allow the solvent to slowly evaporate over two days. The PXRD diffractogram of the resulting crystals of remdesivir maleate Form APO-1 is shown in FIG. 5.

### Example 6: Preparation of Remdesivir Oxalate Form APO-1

To remdesivir free base (5 mg) in acetone (250 μL) was added oxalic acid (0.80 mg). The clear solution was temperature cycled for 16 hours after which the solvent was evaporated. The PXRD diffractogram of the resulting crystals of remdesivir oxalate Form APO-1 is shown in FIG. 6.

### Example 7: Preparation of Remdesivir Oxalate Form APO-II

To remdesivir free base (20 mg) in acetone (1 mL) was added oxalic acid (3.35 mg). The clear solution was covered with a tissue and set aside to allow the solvent to slowly evaporate within one day. The PXRD diffractogram of the resulting crystals of remdesivir oxalate Form APO-II is shown in FIG. 7.

1. A napsylate salt of remdesivir.

2. The napsylate salt of remdesivir of claim 1, wherein the molar ratio of remdesivir to naphthalene-2-sulfonic acid is approximately 1:1.

3. The napsylate salt of claim 2, characterized by a PXRD diffractogram comprising peaks, expressed in degrees 2θ (±0.2°), at 5.1°, 6.5° and 13.1°.

4. The napsylate salt of claim 3, further comprising at least three peaks in the PXRD diffractogram, expressed in degrees 2θ (±0.2°), selected from the group consisting of: 4.5°, 9.0°, 10.0°, 11.5°, 13.60, 15.3°, 16.4°, 17.2°, 20.2° and 24.3°.

5. (canceled)

6. (canceled)

7. The napsylate salt of claim 1, characterized by a DSC thermogram comprising an endothermic peak with a peak onset at approximately 170° C. and a peak maximum at approximately 176° C.

8. (canceled)

9. A tosylate salt of remdesivir.

10. The tosylate salt of remdesivir of claim 9, wherein the molar ratio of remdesivir to p-toluenesulfonic acid is approximately 1:1.

11. The tosylate salt of claim 10, characterized by a PXRD diffractogram comprising peaks, expressed in degrees 2θ (±0.2°), at 5.5°, 7.7° and 13.1°.

12. The tosylate salt of claim 11, further comprising at least three peaks in the PXRD diffractogram, expressed in degrees 2θ (±0.2°), selected from the group consisting of: 4.5°, 6.5°, 9.1°, 9.8°, 16.0°, 16.6°, 17.1°, 17.5°, 18.4° and 20.0°.

13. The tosylate salt of claim 11, further comprising peaks in the PXRD diffractogram, expressed in degrees 2θ (±0.2°), at 4.5°, 6.5°, 9.1°, 9.8°, 16.0°, 16.6°, 17.1°, 17.5°, 18.40 and 20.00.

14. (canceled)

15. The tosylate salt of claim 9, characterized by a DSC thermogram comprising an endothermic peak with a peak onset at approximately 168° C. and a peak maximum at approximately 171° C.

16. (canceled)

17. A salt of remdesivir selected from the group consisting of:
a) a hydrochloride salt of remdesivir;
b) a phosphate salt of remdesivir;
c) a maleate salt of remdesivir, characterized by a PXRD diffractogram comprising peaks, expressed in degrees 2θ (±0.2°), at 10.7°, 12.8°, and 16.3°; and
d) an oxalate salt of remdesivir.

18. The hydrochloride salt of remdesivir of claim 17, wherein the molar ratio of remdesivir to hydrochloric acid is approximately 1:1.

19. The hydrochloride salt of claim 18, characterized by a PXRD diffractogram comprising peaks, expressed in degrees 2θ (±0.2°), at 9.2°, 16.4° and 19.9°.

20. The hydrochloride salt of claim 19, further comprising at least three peaks in the PXRD diffractogram, expressed in degrees 2θ (±0.2°), selected from the group consisting of: 13.4°, 18.3°, 20.60, 21.6° and 24.2°.

21. (canceled)

22. (canceled)

23. (canceled)

24. The phosphate salt of claim 17, characterized by a PXRD diffractogram comprising peaks, expressed in degrees 2θ (±0.2°), at 13.2°, 14.4° and 23.3°.

25. The phosphate salt of claim 24, further comprising at least three peaks in the PXRD diffractogram, expressed in degrees 2θ (±0.2°), selected from the group consisting of: 11.1°, 11.5°, 16.8°, 17.6° and 30.2°.

26. (canceled)

27. (canceled)

28. (canceled)

29. The maleate salt of claim 17, further comprising at least three peaks in the PXRD diffractogram, expressed in degrees 2θ (±0.2°), selected from the group consisting of: 8.1°, 11.1°, 13.9°, 14.7°, 17.0°, 17.7°, 19.8°, 21.0°, 22.6° and 24.5°.

30. (canceled)

31. (canceled)

32. (canceled)

33. (canceled)

34. (canceled)

35. (canceled)

36. (canceled)

37. The oxalate salt of claim 17, characterized by a PXRD diffractogram comprising peaks, expressed in degrees 2θ (±0.2°), at 5.3°, 7.2° and 16.4°.

38. The oxalate salt of claim 37, further comprising at least three peaks in the PXRD diffractogram, expressed in degrees 2θ (±0.2°), selected from the group consisting of: 8.2°, 8.6°, 11.5°, 14.3° and 19.8°.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)
